(19) United States
(12) Patent Application Publication
(10) Pub. No.: US 2023/0355271 A1
(43) Pub. Date: Nov. 9, 2023

(54) METHODS AND SYSTEMS FOR THERAPEUTIC MOLECULE DELIVERY TO SUBCUTANEOUS OR INTRAPERITONEAL SITES

(71) Applicants: ARIZONA BOARD OF REGENTS ON BEHALF OF THE UNIVERSITY OF ARIZONA, TUCSON, AZ (US); PROCYON TECHNOLOGIES LLC, TUCSON, AZ (US)

(72) Inventors: Klearchos Papas, Tucson, AZ (US); Steven Neuenfeldt, Tucson, AZ (US); Robert Johnson, Estes Park, CO (US); Tatum Quimby, Tucson, AZ (US); Leah Steyn, Tucson, AZ (US); Thomas Loudovaris, Tucson, AZ (US)

(73) Assignees: ARIZONA BOARD OF REGENTS ON BEHALF OF THE UNIVERSITY OF ARIZONA, TUCSON, AZ (US); PROCYON TECHNOLOGIES LLC, TUCSON, AZ (US)

(21) Appl. No.: 18/142,368

(22) Filed: May 2, 2023

Related U.S. Application Data
(60) Provisional application No. 63/337,449, filed on May 2, 2022.

(51) Int. Cl. A61B 17/34; A61M 25/02; A61M 25/00

(52) U.S. Cl. CPC A61B 17/3468; A61M 25/02; A61M 25/0026; A61M 39/0247

(57) ABSTRACT

Cannula and device implantation systems are provided. In some embodiments, cannula members are provided that comprise membranes to prevent and reduce infection. Cannulas of the present disclosure are operable to be provided as indwelling catheters. In some embodiments, systems of the disclosure comprise encapsulation devices for housing cells and providing various therapeutic benefits to a patient or host.

### CROSS-REFERENCE TO RELATED APPLICATIONS

This U.S. Non-Provisional patent application claims the benefit of priority from U.S. Provisional Patent Application Ser. No. 63/337,449, filed May 2, 2022, the entire disclosure of which is hereby incorporated by reference in its entirety.

### FIELD

Embodiments of the present disclosure relate to implantable medical devices including but not limited catheter devices. In some embodiments, implantable encapsulation devices are provided that are operable to house cells, tissue, and/or therapeutic agents and deliver therapeutic effects to a host or recipient of the device. Delivery systems, channels and catheters are provided that are operable to indwell in a patient while reducing risk of infection, extending the life expectancy of the implant, and providing various benefits and means for facilitating the delivery of one or more therapeutic agents or other materials.

### BACKGROUND

The number of patients suffering from Type I and Type II diabetes is estimated to affect about 4.6% of the world's population. Pancreas transplantation and islet transplantation are known methods for treating diabetes. However, pancreas and islet transplantation into diabetic patients is limited to a small percent of patients who might benefit from either procedure due to the lack of available human pancreata or pancreatic islets. With the recent development of insulin secreting cells derived from human stem cells, there is a possibility of treating patients with insulin dependent diabetes through transplantation. However, such cells would be subject to rejection by the immune system of the recipient patient unless immunosuppressive drugs were administered to the patient for the rest of their life. Alternatively, insulin secreting cells could be provided with an immuno-isolating implantable device and placed in the diabetic patient to act as an insulin delivery source.

Since the islet transplantation protocol was established, clinical islet transplantation has been regarded as a treatment method for treating type 1 diabetics. However, the low engraftment success of transplanted islet cells remains a major cause of failure of long-term blood sugar regulation. Upon implantation, it is necessary for islet cells to be successfully engrafted through revascularization and blood flow regulation within a few days after transplantation. However, transplanted islet cells are exposed to a state with low vascular density and insufficient oxygen conditions, making it difficult to achieve normal engraftment of islet cells and the ability to achieve regulated insulin secretion in the patient.

Currently, there are limited means and materials to effectively implement live cell containing immuno-isolation devices in vivo. Limitations associated with supply of adequate oxygen levels to encapsulated cells, sufficient nutrient levels to the encapsulated cells, insufficient vascularization of the implanted device and immune response to the implant, remain barriers to use of cell-containing implantable devices.

Known subcutaneous delivery systems for insulin and other therapeutic agents include cannula-based injection systems. While various transdermal catheter systems are known in the medical industry, such known systems provide for enhanced risk of infection at the implantation site particularly in applications and systems where the catheter is to be integrated into a patient for a long period of time. Other benefits include extending the duration or life expectancy of the catheter over current catheter systems, along with preventing catheter occlusions in the openings for delivering therapeutic agents.

### SUMMARY

Embodiments of the present disclosure provide for an indwelling medical device that is operable to be implanted through the skin of a patient. Such embodiments comprise a catheter member that is operable to heal-in-place as shown and described herein in a biocompatible manner at least at the implantation site and/or interface with the skin of the patient. Catheter members of the present disclosure are operable to be provided in communication with additional components including, but not limited to pumps, implantable devices, and fluid supply reservoirs. The catheter members are preferably operable to deliver one or more therapeutic agents to the patient/host, deliver fluids (liquid or gas), exchange fluids, and/or communicate with additional components. In various embodiments, catheter members of the present disclosure comprise a membrane with pore sizes of between approximately 1 and 100 microns, and more preferably of between approximately 2 and 25 microns. Membranes of the present disclosure facilitate the indwelling or healing-in-place features of the present disclosure. The membrane(s) are operable to induce host cellular ingress and prevent a foreign body response when implanted in the body. Such membranes may be referred to herein as “vascularizing membrane(s)”, “vascularization membrane(s)” or “cell integrating membrane”.

In preferred embodiments, devices of the present disclosure comprise a catheter member encased in a vascularizing membrane. The catheter member and vascularizing member are secured to one another in a manner that enables healing through and below and the skin. Bonding and securing means are provided to prevent or minimize the risk of bacteria entering at the skin-catheter junction or along the catheter under the vascularizing membrane, thus eliminating or at least reducing the risk of tunnel site infections. Bonding and securing means as used herein includes, but is not limited to, the provision of various adhesives or thermal energy such as thermoplastic, silicone, cyanoacrylate, and polymer adhesives provided to secure a membrane to a catheter tube. A preferred embodiment of the present disclosure contemplates thermal energy application to secure or bond a vascularizing membrane to a catheter in a manner to enable host cellular ingress into the pores of vascularizing membrane without a foreign body response along the outside of the catheter.

Embodiments of the present disclosure contemplate heat application to secure a membrane to a substrate (e.g. catheter member). Various shrink tubing and low-melt applications are contemplated to thermally and evenly shrink a membrane to its intended position and reduce or eliminate voids known to increase risks of tunnel site infection. The mechanical bonding of such applications and embodiments further provide a secure connection between adjacent components.

Embodiments of the present disclosure enable long-term implantation and access through the skin, reduce or eliminate a need to exchange catheters, and reduce the burden or need for the patient-host to maintain sterile implantation site, at least in part due to the provision of a vascularization membrane. Catheter members and systems of the present disclosure enable connection for up to two or more implantable devices comprising a lumen for communicating therapeutic agents to the bloodstream of a patient. Catheters of the present disclosure are further operable to deliver chemical agents, bodily fluids, electrical leads, electrical signals, gases (e.g. oxygen) to or from the host.

In some embodiments, catheters of the present disclosure are provided in communication with and/or comprise a small diameter tube comprising multiple holes, ports, slots or breaches for delivery of a therapeutic agent (e.g. insulin) to the host at a subcutaneous or intraperitoneal site. Such catheters may be occasionally referred to herein as an “insulin delivery catheter(s)”. Therapeutic agents are operable to be pumped or injected through the catheter until a specific amount is pushed or diffused through the holes, ports, slots or breaches in the catheter. The holes, ports or slots are contemplated as comprising a small pore membrane covering the holes to avoid occlusion and maintain transport properties and abilities. The small pore membrane is contemplated as comprising pore sizing of between approximately 0.01 and 5 microns and more preferably of about 0.1 to 2 microns. It is further contemplated that, in some embodiments, a vascularizing membrane is provided that covers the small pore membrane to enable host cellular ingress, molecular transport, vascularization, healing along the catheter and through the skin, and prevention of a foreign body response. In such embodiments, the combination of the vascularizing membrane and the small pore membrane provides for the formation of host vascular structures to enable faster uptake of the therapeutic agent along at least a portion of the length of the catheter. Embodiments of the present disclosure reduce or eliminate the need for a host or patient to frequently change catheters or infusion site devices and reduce or eliminate the risk of a foreign body response.

In some embodiments, catheters of the present disclosure are provided with multiple lumens in communication with and/or comprising holes, ports, slots or breaches for delivery of one or more therapeutic agents (e.g. insulin and glucagon) to the host at a subcutaneous or intraperitoneal site. In such embodiments, the combination of the multiple lumens with holes, ports, slots or breaches are unique to a particular lumen and provide the function of counter regulation of therapeutic agents such as, insulin and glucagon, or the independent delivery of multiple therapeutic agents.

In some embodiments, catheter members of the present disclosure are provided in combination with or communication with a long-term implantable device including, for example, an immunoisolation device for cellular encapsulation. Such implantable devices are contemplated as comprising but are not limited to those shown and described in U.S. Patent Application Publication Nos. 2019/0328289 to Papas, 2020/0281709 to Papas, 2020/0063085 to Papas, 2019/0224377 to Papas, 2020/0054257 to Papas, 2021/0401564 to Neuenfeldt et al., and International Application PCT/US2021/057526 to Papas, which are hereby incorporated by reference in their entireties. In some embodiments, the catheter and associated implantable device(s) are encased in or otherwise comprise a vascularizing membrane to enable vascularization of the encapsulation device prior to cellular implantation and creates an optimal environment around the implantable device to support transplanted cells or tissues without causing a foreign body response prior to cellular implantation. It is contemplated that a vascular bed is provided and serves as a means of delivering nutrients to cells within the implantable device and as a means for absorbing therapeutic molecules secreted by the cells and delivering the molecules to the rest of the body. In various embodiments, oxygen delivery systems and features are provided to convey oxygen to the cell chamber(s) prior to or following cellular implantation.

In some embodiments, the catheter and/or associated implantable device(s) are encased in or otherwise comprise a gradient membrane to enable vascularization and provide an immuno-isolating barrier between the host tissue and implanted device(s).

In some embodiments, systems of the present disclosure comprise oxygen delivery features. In certain embodiments, catheter and/or implantable device systems comprise oxygen delivery provided by an oxygen diffuser. For example, it is contemplated that an oxygen diffuser is provided in an implantable device adjacent or proximal to cellular contents of the device. Oxygen is contemplated as being delivered into a chamber surrounded by separate chambers containing cells, for example. Alternatively, oxygen is delivered to a diffuser provided between cell chambers. Diffusers of the present disclosure are contemplated as comprising features to diffuse oxygen and/or other nutrients to cells. In some embodiments, a diffuser is contemplated that comprises a hydrophobic or super-hydrophobic membrane to separate the transplanted cells from an oxygen course while preventing ingress of fluid(s) to the means for supplying or venting oxygen to optimize transport and prevent occlusions in the oxygen supply or venting. Diffusers of the present disclosure are further contemplated as comprising a semi-permeable material (e.g. silicone) to transport oxygen to cells. Such a diffuser is contemplated in some embodiments as comprising an insert such as a wafer. Diffusers are also contemplated as comprise tubes or channels wherein at least a portion of the tube or channel is gas permeable.

Another aspect of the present disclosure is to provide an over-pouch member in the form of a physical container. In some embodiments, the container comprises a solution to support a cellular encapsulation device. The over-pouch member is contemplated as comprising a super-hydrophobic membrane to enable degassing of volatiles such as ethylene oxide gas and oxygen without the loss of fluid(s) such as media from the over-pouch.

Membranes of the present disclosure are contemplated as comprising various materials, including those deemed appropriate by a person skilled in the art for an implantable medical device. For example, membranes of the present disclosure are contemplated as being prepared from a polymeric material. In such embodiments, the single layer gradient membrane is prepared from such polymeric materials as: polysulfone, polyarylethersulfone (PAES), polyethersulfone (PES), cellulose ester (cellulose acetate, cellulose triacetate, cellulose nitrate), nanocellulose, regenerated cellulose (RC), silicone, polyamide (nylon), polyimide, polyamide imide, polyamide urea, polycarbonate, ceramic, titanium oxide, aluminum oxide, silicon, zeolite (alumosilicate), polyarylonitrile (PAN), polyethylene (PE), low density polyethylene (LDPE), polypropylene (PP), polypiperazine amide, polyethylene terephthalate (PET), polycarbonate (PC), polyurethane, and any complex or mixtures thereof. In particular embodiments, a single layer gradient membrane comprises of a polymeric material comprising polytetrafluoroethylene (PTFE). In certain preferred embodiments, PTFE is provided for at least a vascularizing layer of devices of the present disclosure. Additional materials are contemplated as being provided in membranes and implants of the present disclosure in addition to or in lieu of PTFE.

In some embodiments, a gradient membrane is provided that comprises an electro-spun polymeric membrane, such as an electrospun PTFE membrane that is applied directly to a surface, such as a surface of an implantable medical device or a catheter tube. Implantable medical devices of the present disclosure are contemplated as comprising an internal chamber of live cells. No separate assembly steps are required to provide a protective layer/film to an internal chamber of an implantable medical device in which live cells may be contained, as the single layer gradient membrane is capable of protecting the cells from immune attack, while simultaneously permitting nutrient flow/oxygen to contained live cells, owing to the appropriate gradient pore size provided by the single layer gradient membrane. Single layer gradient membranes of the present disclosure also provide for a slightly larger pore size within the membrane region extending to the other surface (e.g., outer surface) of the single layer membrane, thus providing a surface suitable for vascularizing the outer surface of an implantable medical device or an indwelling catheter in a host.

In various embodiments, membranes are formed with phase inversion, interfacial polymerization, solution coating and/or phase deposition methods. These and other processes are described in Baker (Baker, R. Membrane Technology and applications. John Wiley & Sons, 2004), which is hereby incorporated by reference in its entirety.

In various embodiments, electrospinning is provided as a process to control fabricating a fibrous mat of changing and defined density in a single layer membrane construction.

It is an aspect of the present disclosure to provide materials and processes that provide for the elimination of delamination problems of prior fabricated techniques having a bi-layer membrane structure. In addition, the method by which the single layer, gradient membranes are prepared are preferable to other 2-step processes, that require a separate lamination and/or fusing step between two separately fabricated membranes, such as that described in U.S. Pat. No. 6,060,640, which is hereby incorporated by reference in its entirety.

In various embodiments, implantable medical devices are provided that comprise at least one surface upon which a single layer membrane material having a gradient structure is applied. The surface is contemplated as comprising the surface of a catheter or an implantable medical device, such as an implantable device that has a lumen comprising living cells (e.g. stem cells).

In various embodiments, membranes are employed as coatings on any or all surfaces of a catheter and/or an implantable medical device. Some surfaces of a catheter and/or implantable device may be devoid of a membrane. For example, surfaces at which fibrotic mass formation is not a significant occurrence are contemplated as being devoid of membranes. Additional surfaces that are devoid of a membrane include, for example, surfaces at a sonic weld joint on an access port of an implantable medical device and a section of catheter tubing that will not reside within the skin or body of the patient.

In one embodiment, membranes are provided that reduce overall fibrosis and comprises pores having a size of about 0.01 to about 100 micron (or, from about 0.01 or about 5 micron to about 15 micron). In some embodiments, an implantable medical is provided that comprises a lumen comprising living cells. The single layer gradient membrane comprises a pore size that does not interfere with the passage of molecules (such as insulin produced by contained islet cells) out of a lumen chamber (having its own chamber lining), and out of the implantable medical device into the body. In this regard, the membrane is sufficiently thin so as to allow rapid diffusion of molecules out of the implantable medical device. As another example, a single layer gradient membrane is provided on some surfaces of a component of a multi-component implantable medical device and not on other surfaces.

In certain embodiments, systems are provided that comprise a surface having a membrane, such as a membrane comprising a polymeric material. By way of example, the polymeric material is contemplated as comprising PTFE, where the PTFE membrane comprises a gradient of pore sizes. This single layer PTFE gradient membrane is provided to the external surface of the implantable medical device system. The outer side (host vasculature inter-facing) of the PTFE gradient membrane enables cellular ingress (greater than 1 micron to about 15 micron), and the PTFE gradient membrane titrates down in relative pore size to an appropriate size that would prohibit cellular ingress (about 0.1 micron to about 1 micron) into the cell-containing inner chamber of the implantable medical device. The pore size of the PTFE gradient membrane renders the implantable medical device immuno-isolating for the implanted cells.

In further embodiments, implant systems comprise one or more surfaces with an electrospun PTFE gradient membrane combining immunoisolation and vascularization features as described above. An electrospun PTFE multielement layer comprises relatively larger fibers, of a size sufficient to inhibit fibroblast layer formation. This feature may take the form of a final, outer gradient layer comprising multiple strands to form thick fibers of about 25 to about 200 micron in diameter. With such larger fibers randomly oriented on the outer surface of the gradient membrane, the layer serves as a surface to inhibit fibroblasts from forming a fused fibrotic layer.

In various embodiments, the present disclosure provides implantable devices having a number of improved characteristics and features. In some embodiments, an implantable device is provided that possesses a unique configuration that facilitates a maximization of surface area available for vascularization by a host animal. The configuration of the implant device, in some embodiments comprises a multi-component structure, comprising one or more individual element members and a hub and/or a manifold, wherein the individual pod elements are in communication with the hub and/or manifold. In this regard, means are provided that permit multiple of the individual element members of the device to communicate with at least one common component of the device, such as a hub or a manifold. In this manner, and where the individual member element comprises an internal lumen, access to the lumen of each individual element member and the hub and/or manifold is provided.

In some embodiments, it is contemplated that multiple implantable encapsulation devices are provided in a patient. In such embodiments, the devices are contemplated as being connected to a single catheter, including a catheter in accordance with the embodiments shown and described herein, with one or more bifurcations to divert and deliver a supply of fluid (e.g. insulin) to the devices. In other embodiments, it is contemplated that a dedicated catheter (including, for example, those shown and described herein) is provided for each individual implantable device. Systems and associated methods of the present disclosure in accordance with these embodiments contemplate providing a fluid (e.g. insulin) in a specific amount over a period of time. It is contemplated that an implantation site and associated surrounding tissue may experience a diminished or adverse response over a period time and/or upon delivery of a certain amount of fluid or therapeutic agent. It is thus contemplated that fluid delivery is switched or diverted to at least one additional implantable device that is preferably spaced apart from the first device. In some embodiments, the devices are spaced apart by 1-50 millimeters. In further embodiments, it is contemplated that implantable devices are spaced apart by a greater amount. For example, it is contemplated that two implantable encapsulation devices each comprising at least one lumen for insulin deliver are provided. A first device is implanted in a patient's left arm and a second device is implanted in a patient's right leg, with no limitation provided as to a specific distance or amount of separation. When a first implantation location demonstrates a reduced response (e.g. from a glucose or oxygen monitor or other testing), fluid delivery can be modified such that fluid is then delivered to the second site as opposed to the first. It is further contemplated that more than two such devices are provided. For example, three or more (e.g. twenty) devices can be implanted in a patient.

International Application Nos. PCT/US2017/060036 to Papas, PCT/US2017/060034 to Papas, PCT/US2017/060041 to Papas, and PCT/US2017/060043 to Papas relate to encapsulation devices and are each incorporated by reference in their entireties herein for all purposes.

Devices of the present disclosure comprise various materials, including those deemed appropriate by a person skilled in the art for an implantable medical device. For example, membranes of the present disclosure are contemplated as being prepared from a polymeric material. In such embodiments, the single layer gradient membrane is prepared from such polymeric materials as: polysulfone, polyarylethersulfone (PAES), polyethersulfone (PES), cellulose ester (cellulose acetate, cellulose triacetate, cellulose nitrate), nanocellulose, regenerated cellulose (RC), silicone, polyamide (nylon), polyimide, polyamide imide, polyamide urea, polycarbonate, ceramic, titanium oxide, aluminum oxide, silicon, zeolite (alumosilicate), polyarylonitrile (PAN), polyethylene (PE), low density polyethylene (LDPE), polypropylene (PP), polypiperazine amide, polyethylene terephthalate (PET), polycarbonate (PC), polyurethane, and any complex or mixtures thereof. In particular embodiments, a single layer gradient membrane comprises a polymeric material comprising polytetrafluoroethylene (PTFE). In certain preferred embodiments, PTFE is provided for at least a vascularizing layer of devices of the present disclosure. Additional materials are contemplated as being provided in membranes and implants of the present disclosure in addition to or in lieu of PTFE.

The advantages of the presently disclosed immune-isolation catheters and implantable devices include a maximization of surface area presented by the device available for vascularization by a host. In particular, implantable devices or portions thereof that comprise an immuno-isolation device present surface area that may be vascularized by the host when implanted. This structure maximizes vascularization of the device as a whole in the animal.

Various concepts disclosed herein may be provided in combination with one another even if such combination is not specifically depicted or described.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

### DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the inventive concepts disclosed herein.

The accompanying drawings, which are incorporated in and constitute a part of the specification, illustrate embodiments of the disclosure and together with the general description of the disclosure given above and the detailed description of the drawings given below, serve to explain the principles of the disclosure.

FIG. 1A is an elevation view of a catheter according to one embodiment of the present disclosure.

FIG. 1B is a plan view of the catheter according to the embodiment of FIG. 1A.

FIG. 1C is a perspective view of a catheter according to one embodiment of the present disclosure.

FIG. 1D is a cross-sectional elevation view along the longitudinal length of a catheter according to one embodiment of the present disclosure.

FIG. 2 is an elevation view of a catheter according to an embodiment of the present disclosure in communication with an implantable device as contemplated by various embodiments of the present disclosure.

FIG. 3 is a cross-sectional elevation view of a catheter according to one embodiment of the present disclosure and taken at line 3-3 of FIG. 1A.

FIG. 4 is a partial cut-away view of a catheter according to one embodiment of the present disclosure.

FIG. 5 is an elevation view of a catheter in communication with an oxygen device and an implantable device according to one embodiment of the present disclosure.

FIG. 6A is an elevation view of a catheter according to an embodiment of the present disclosure.

FIG. 6B is a plan view of a catheter according to an embodiment of the present disclosure.

FIG. 7 is a plan view of a catheter according to one embodiment of the present disclosure provided in communication with a plurality of implantable devices.

FIG. 8A is a plan view of an encapsulation device according to one embodiment of the present disclosure and contemplated for use with catheter systems of the present disclosure.

FIG. 8B is a plan view of an encapsulation device according to one embodiment of the present disclosure and contemplated for use with catheter systems of the present disclosure.

FIG. 8C is a plan view of an encapsulation device according to one embodiment of the present disclosure and contemplated for use with catheter systems of the present disclosure.

FIG. 8D is a plan view of an encapsulation device according to one embodiment of the present disclosure and contemplated for use with catheter systems of the present disclosure.

FIG. 8E is a plan view of an encapsulation device according to one embodiment of the present disclosure and contemplated for use with catheter systems of the present disclosure.

FIG. 9A is an elevation view of a catheter in communication with an implantable device according to one embodiment of the present disclosure.

FIG. 9B is a cross-sectional elevation view taken at line A-A of FIG. 9A.

FIG. 10 is a plan view of a catheter system and related components according to an embodiment of the present disclosure.

The drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

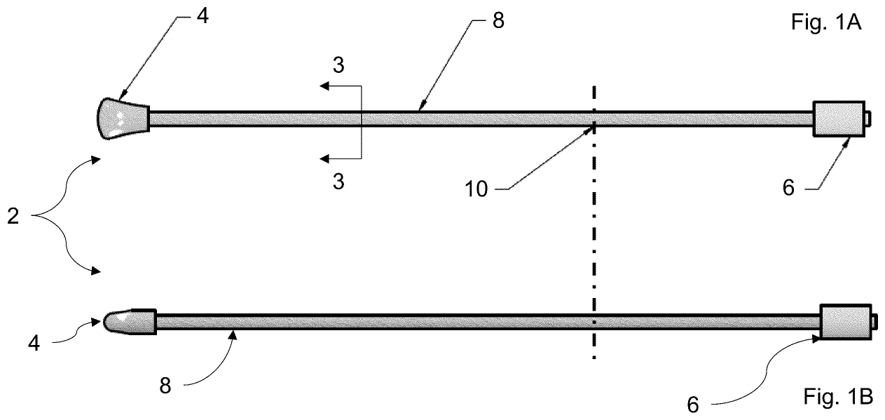

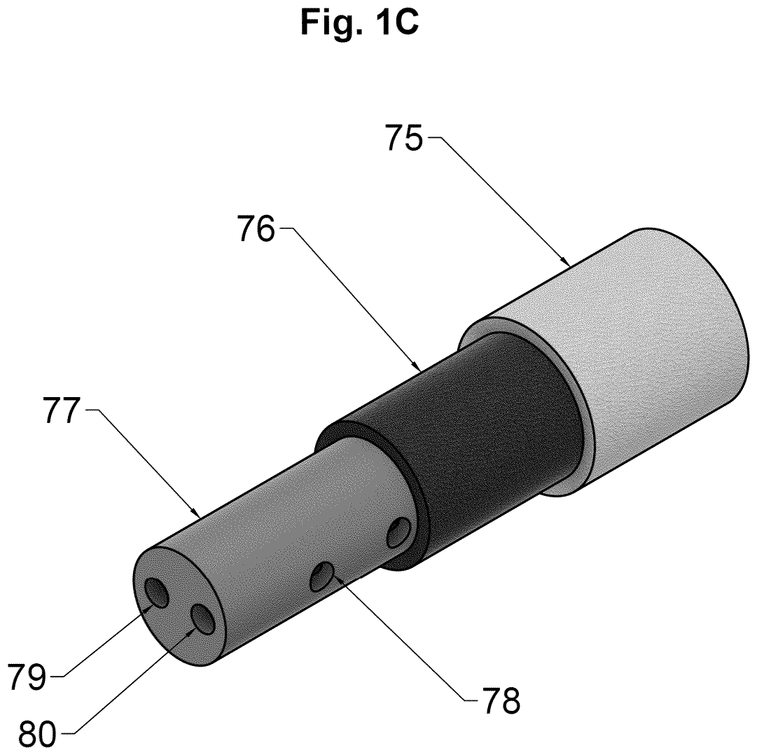

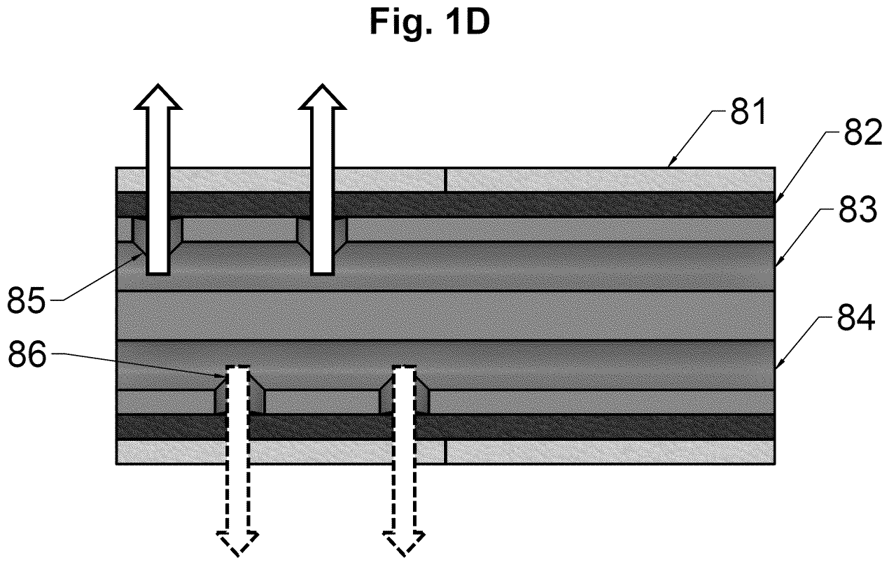

FIGS. 1A-1B are top and side views of a catheter 2 according to one embodiment of the present disclosure. As shown, the catheter 2 comprises a device that is operable to be implanted in or through the skin of a patient. While preferred embodiments of the present disclosure contemplate catheters that are operable to extend through a patient's skin, the present disclosure is not limited to such embodiments. Indeed, catheters and catheter features as shown and described herein are contemplated as being operable to and useful for various catheter applications. For example, catheters and catheter features of the present disclosure may be provided or useful in urinary catheter applications and other fluid exchange applications.

In some embodiments, the catheter 2 is operable to and intended to indwell in a patient and remain in the patient for an extended time (an amount of time longer than what is needed to deliver a single dose of therapeutic agent, for example). As shown, the catheter 2 comprises a first end, a second end, and a length extending therebetween. In some embodiments, the first end is contemplated as comprising a first connection member 4 that is operable to connect to and communicate with various additional devices (not shown in FIGS. 1A-1B). In alternative embodiments, the first of the device does not comprise a connection member 4 but rather comprises an open-ended channel that is operable to communicate fluid(s) to and from a patient. In some embodiments, the first connection member 4 comprises one or more known connection members such as luer lock devices or similar connecting components to secure the first end of the catheter to another component (e.g. an implantable device). A second end of the catheter comprises a second connection member 6 that is operable to connect to and place the catheter 2 in fluid communication with one or more additional components preferably residing external to the patient. For example, the second connection member 6 is contemplated as being operable to connect the catheter 2 to one or more of an insulin supply, an insulin pump, an oxygen generator, an oxygen diffuser, a source of cleaning or flushing fluid (e.g. saline), and a supply of one or more therapeutic agents. In some embodiments, systems and catheters of the present disclosure comprise a vent. For example, an ex-vivo vent is contemplated as being provided to provide a means and pathway to expel excess gas (e.g. oxygen).

The catheter 2 comprises an elongate channel that is operable to deliver fluid (i.e. liquid and/or gas) to a patient. An outer portion of the catheter 2 comprises a vascularization membrane 8. The vascularization membrane comprises pores sizes of between approximately 1 and 100 microns, and preferably of between about 2 and 25 microns. As used herein, the term “micron” is used in the singular and plural to refer to micrometer and/or micrometers. The vascularization membrane 8 preferably comprises a layer or structure that allows for ingrowth of a patient's vasculature while preferably preventing ingrowth of fibrotic tissue. In some embodiments, the vascularizing member extends along the entire length of the catheter. In other embodiments, the vascularizing layer 8 extends along only a portion of a length of the catheter. FIGS. 1A-1B illustrate a line or point 10 of maximum insertion to which the catheter is to extend into a patient. It is contemplated that in some embodiments, the vascularizing member extends from the first end to the point 10 of maximum insertion or just beyond that point. Vascularizing membranes of the present disclosure are also contemplated as extending around an entirety of a circumference or periphery of the catheter. In alternative embodiments, however, it is contemplated that the vascularizing membrane extends around less than an entirety of the circumference of the catheter. In various embodiments, the vascularization membrane 8 comprises a thickness of between approximately 10 and 200 microns. No limitation with respect the overall length of the catheter(s) and any associated vascularization membrane(s) are provided herewith and one of ordinary skill in the art will recognize that this length may vary based on the application, patient, etc. The bonding systems and methods, as well as the tissue growth provided by and within the vascularization membrane(s) of embodiments of the present disclosure provide for a physical barrier for foreign bodies and materials that prevent an infection risk.

FIG. 1C is a perspective view of an alternative embodiment that comprises a vascularizing membrane 75 and an immuno-isolating membrane 76. A catheter 77 is provided that comprise one or more holes, ports, slots or breaches 78 in communication with one or more lumens 79, 80 within the catheter. the vascularization membrane 75 comprises a thickness of between approximately 10 and 200 microns. The immuno-isolation membrane 76 has a thickness of between approximately 1-100 microns with pore sizes of between approximately 0.01 micron and 1 micron to prevent cellular ingress from the host into the holes, ports, slots or breaches 78 of the catheter 77.

FIG. 1D is a cross-sectional elevation view of an alternative embodiment of a catheter 84. FIG. 1D shows a catheter 84 with two lumens 83, 84 or channels in communication with separate associated holes, ports, slots or breaches 85, 86. The lumens 83, 84 and associated holes, ports, slots or breaches 85, 86 are isolated from each other. The catheter 84 may be encased in an immuno-isolating membrane 82 and vascularizing membrane 8. The catheter and its multiple lumens 83, 84 are operable to deliver multiple fluids including, for example, simultaneously delivering insulin and oxygen. It is also contemplated that one lumen is provided as a delivery lumen while a second lumen is provided as a vent or egress lumen.

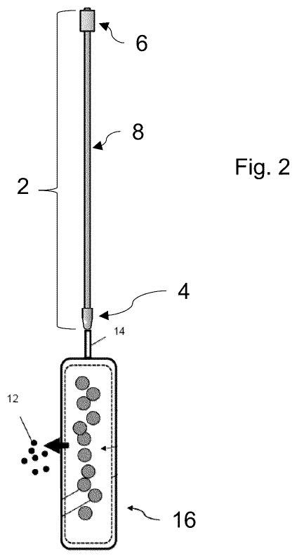

FIG. 2 is a view of a catheter device 2 according to embodiments of the present disclosure in combination with an implantable device 16. Catheters 2 of the present disclosure are contemplated for use in various applications and with various devices. For illustrative purposes, FIG. 2 depicts a catheter in communication with an implantable cell encapsulation device that is operable to house cells (e.g. insulin producing islet cells) and release non-cell factors or molecules 12 into the body. It is contemplated that devices such as that shown in FIG. 2 may require a pathway and/or connection member for communicating fluid(s) to and from the device 16. Accordingly, it is contemplated that catheter devices and features of the present disclosure are connected to the device 16 and wherein the device is implanted subcutaneously and at least a portion of the catheter 2 extends external to the patient. The second connection 6 of the catheter 2 is operable to be connected to a supply of insulin, oxygen, or other material. At least a portion of the length of the catheter 2 and preferably at least a portion of the implantable device 16 is coated, wrapped, or surrounded by a vascularization membrane 8. A first connection member 4 and port 14 of the device 16 are further contemplated as comprising a vascularization membrane. While FIG. 2 depicts a catheter 2 in communication with an implantable device 16, alternative embodiments and catheter arrangements are contemplated. For example, it is contemplated that catheters of the present disclosure are contemplated for use when not connected to an implantable device. In some embodiments, catheters comprise at least one open end and are implanted through or partially into a patient's skin to drain or irrigate an infected area (for example). In such embodiments, the catheter 2 is allowed to indwell in the patient while minimizing the risk of further infection.

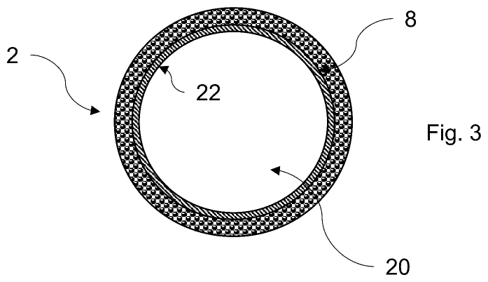

FIG. 3 is a cross-sectional elevation view of a catheter 2 taken at line 3-3 of FIG. 1A. As shown, the catheter 2 comprises a catheter tube 20 having a central channel or lumen through which fluid is allowed to flow. The catheter tube 20 is surrounded by a vascularization membrane 8 along at least a portion of a length of the catheter tube 20. The vascularization membrane 8 is contemplated as comprising a layer of PTFE, polymer, silicone, or laminate, for example, with pore sizes preferably between approximately 2 and 25 microns. A bonding or adhesive layer 22 is provided which is contemplated as comprising one or more of a thermoplastic, silicone, cyanoacrylate, polymer, or similar adhesive. A catheter tube 20 comprising a lumen is provided that comprises at least one of a polymer, thermoplastic, silicone, or similar tube or channel. The tube and the vascularization membrane 8 are adhered or secured together by the adhesive layer 22.

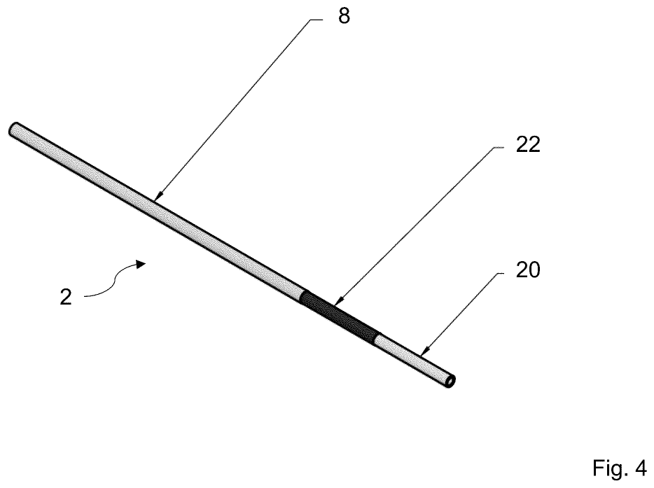

FIG. 4 is a partial cut-away view of the catheter 2 of FIG. 3. As shown, the catheter 2 comprises an outer vascularization membrane 8 secured to a catheter tube 20 by an adhesive layer 22. Although certain embodiments of the present disclosure depict a catheter member having a substantially circular cross-section, the present disclosure is not limited to such embodiments. Indeed, it is contemplated that catheters of the present disclosure may comprise various arrangement and cross-sectional shapes.

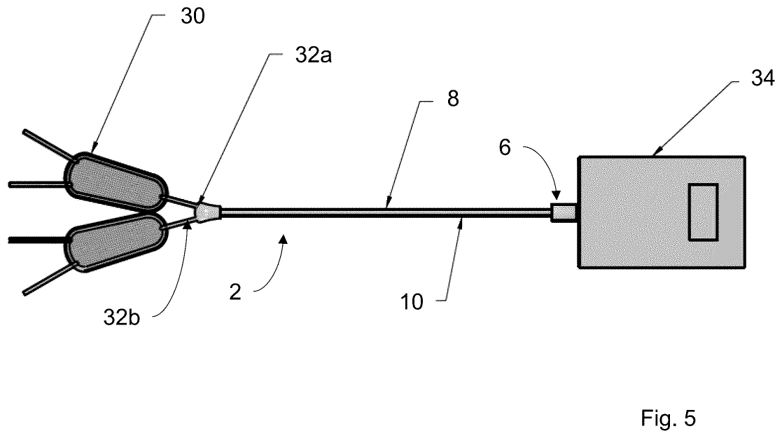

FIG. 5 is a plan view of a catheter 2 provided in communication with additional components of the present disclosure. Specifically, the catheter 2 is provided in communication with first and second implantation devices 30. The devices 30 are contemplated as comprising, but are not limited to, implantable cell encapsulation devices as shown and described in U.S. Patent Application 2021/0401564 to Neuenfeldt, the entire disclosure of which is hereby incorporated by reference. A first connecting component 4 is in communication with first and second ports 32a, 32b such that the lumen of the catheter 2 is in communication with the implantable devices. A second end of the device is provided in communication with an additional component shown an oxygen generator 34 in FIG. 5. The oxygen generator is operable to, for example, provide a flow of oxygen through the catheter 2 and to the implantable device(s) including internal volumes or lumen(s) of the devices 30. A point of maximum skin/body penetration 10 is illustrated in FIG. 5. As shown, the oxygen generator is provided external to a patient (not shown in FIG. 5).

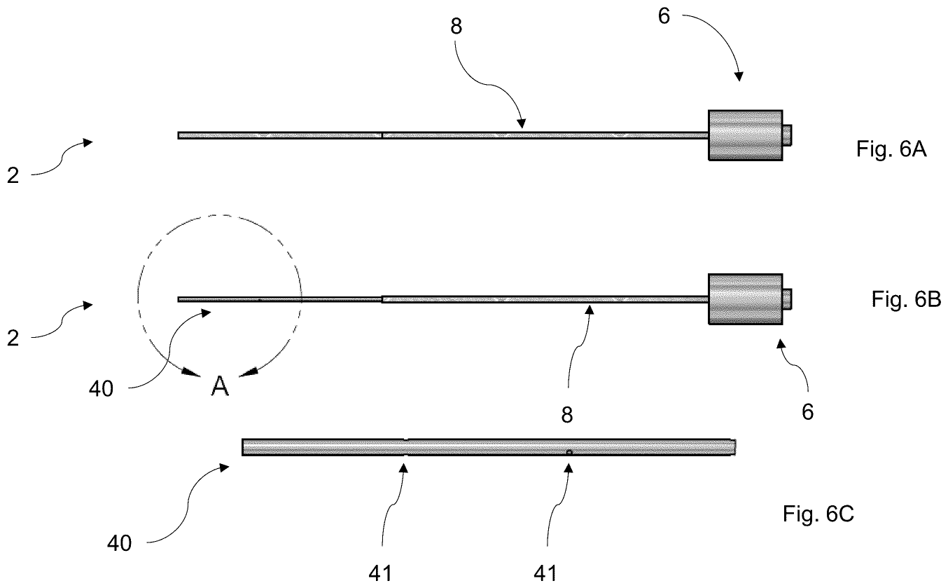

FIG. 6A is a plan view of a catheter 2 according to an embodiment of the present disclosure. As shown, the catheter 2 comprises a vascularization membrane 8 surrounding a catheter channel. A connecting member 6 is shown, preferably to connect the catheter 2 to additional components and devices external to the body. FIG. 6B is an elevation view of the catheter 2 of FIG. 6A.

The catheter channel or tubing comprises an adhesive coating to bond layers of the device. The adhesive coating is contemplated as comprising at least one of a polymer, a thermal plastic, silicone, and similar materials. A distal end 40 of the catheter 2 is contemplated as comprising through holes, slots or breaches to allow for the passage and communication of therapeutic agents. Such agents may be communicated directly into the patient's body and/or may be provided to an implantable device as shown and described herein. In some embodiments, including that shown in FIGS. 6A-6B, a lumen or channel of the catheter 2 is surrounded by a vascularization membrane 8 and the catheter channel is impermeable along at least a portion of its length. For example, and as shown in FIG. 6B, cells, fluids, therapeutic agents, etc. are allowed to exit the catheter at or proximal to the distal end 40 of the catheter and are not able to transmit through other portions of the catheter channel. The device of FIG. 6B further comprises an immunoisolation membrane provided between the vascularization membrane 8 and the catheter tubing.

Embodiments of the present disclosure, including but not limited to those shown in FIGS. 6A-6C, comprise catheter members with an internal channel that is operable to allow egress of therapeutic agents and other materials at or proximal to a distal end of the catheter in a subdermal manner. Proximal portions of the catheter including a length of portion of the catheter that is intended to reside in or adjacent to a patient's dermis comprise membranes as shown and described herein, and the length of portion proximal to the dermis do not comprise egress ports.

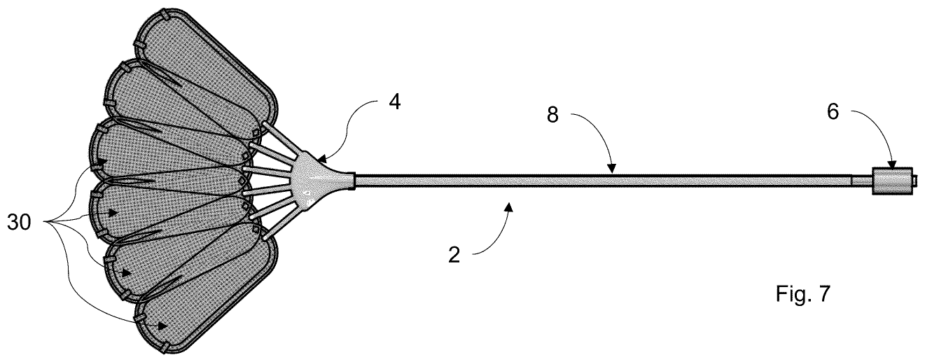

FIG. 7 is a plan view of a catheter system comprising a catheter 2 with a vascularization membrane 8 provided around a catheter tube. A first end of the catheter 2 comprises a connection member 4 depicted as a manifold operable to provide a plurality of implantation devices 30 in communication with the catheter channel. A second end of the catheter 2 comprises a second connection member 6 that is operable to be secured to and/or receive various additional devices including, but not limited to, insulin sources, pumps, gas delivery means (e.g. oxygen pumps or diffusers), etc.

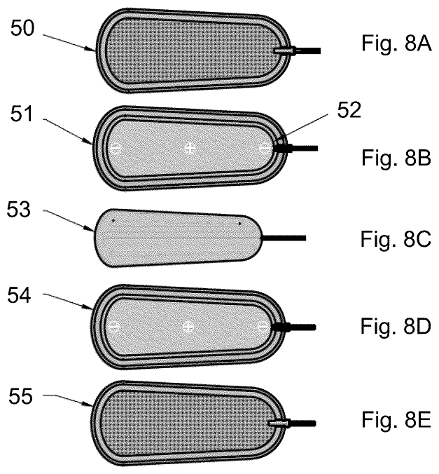

FIG. 8A is a plan view of a cellular encapsulation device 50 with an internal diffuser according to an embodiment of the present disclosure, the components thereof being shown and described with respect to FIGS. 8B-8E. The device 50 comprises upper and lower lumens and a diffuser provided therebetween. FIG. 8B illustrates the device 50 with an upper lumen removed and a diffuser 52 is contained within a middle area and enclosed an immunoisolation membrane. In some embodiments, the immunoisolation membrane comprises a superhydrophobic material. FIG. 8C illustrates the diffuser 53 in isolation (i.e. without upper and lower cellular encapsulation device layers).

In various embodiments, one or more diffusers 53 are provided. The diffusers 53 are contemplated as being provided as wafers or wafer-type devices that preferably comprises an inert, non-toxic material such as silicone and similar materials(s). The diffuser 53 is contemplated as comprising one or more gas conduits for supplying fluid (e.g. oxygen gas) to the device and/or the contents of the device. In some embodiments, one or more diffusers are provided to occupy a volume of space within a device. For example, in some embodiments, a diffuser is provided to fill an inner volume or space and render that volume unusable for cells, fluids, and therapeutic agents. The provision of such diffusers, wafer, and/or spacers are provided to adjust a ratio of the external surface of the device to the usable internal volume. For example, in applications where vascularized surface area is to be maximized and the internal volume available for insulin to be received is to be reduced, the wafer(s) 53 is provided to fill or occupy space and achieve the intended result.

FIG. 8D illustrates a lower cellular encapsulation device 54 without the upper encapsulation device or diffuser. FIG. 8E illustrates a lower cellular encapsulation device 55 without the upper device, diffuser, or immunoisolation membrane layers. The components of FIGS. 8B-8E are contemplated as being stacked or layered to form the device of FIG. 8A.

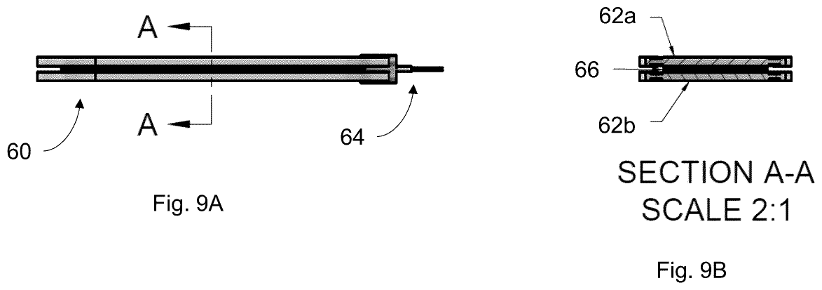

FIG. 9A is an elevation view of an implantable device 60 contemplated for use with embodiments of the present disclosure. As shown, the device 60 comprises first and second cellular encapsulation devices 62a, 62b. It is contemplated that in certain embodiments, the first and second devices 60a, 60b comprise substantially the same construction. A port 64 is provided. The port is contemplated as being operable to receive cells for loading into the encapsulation devices 62a, 62b and/or may connect to one or more catheter devices as shown and described herein. FIG. 9B is a cross-sectional elevation view of the device 60 of FIG. 9A. As shown, a diffuser 66 is provided between the first and second encapsulation devices 62a, 62b. The diffuser is operable to provide oxygen to the devices and cells (for example) housed within the devices. In some embodiments, the diffuser comprises a gas permeable member such as a gas permeable silicone tubing.

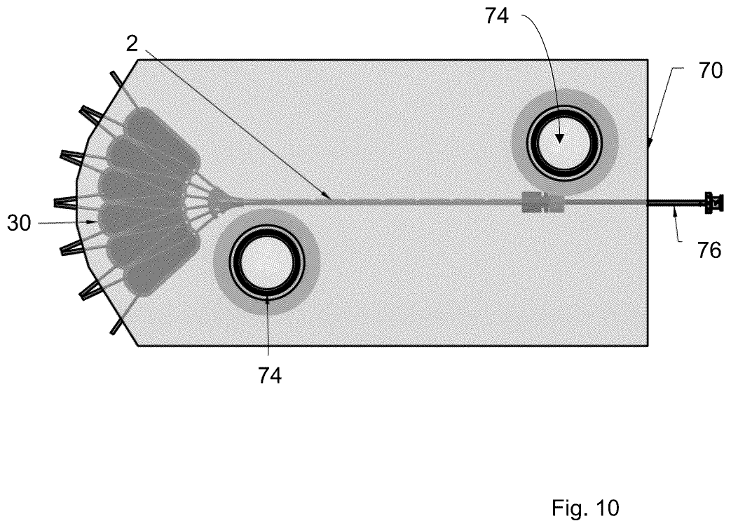

FIG. 10 is a plan view of a system 70 according to an embodiment of the present disclosure. As shown, the system 70 comprises a catheter 2 in communication with a plurality of cellular encapsulation devices 30. A containment vessel or over-pouch 72 is provided and the catheter 2 and devices 30 are provided within the over-pouch 72. The over-pouch is contemplated as comprising and housing a solution to support the cellular encapsulation device(s). One or more openings 74 are provided to allow for ingress and egress of a solution to be provided within the over-pouch 72. At least one port 76 is provided to allow at least the catheter 2 to communicate with components, features, etc. external to the over-pouch 72. The over-pouch 72 is contemplated as a means for preparing, servicing, and maintaining devices such as cellular encapsulation devices and heal-in-place catheters in vitro.

The examples set forth above are provided to give those of ordinary skill in the art a complete disclosure and description of how to make and use the embodiments of the methods for prediction of the selected modifications that may be made to a biomolecule of interest, and are not intended to limit the scope of what the inventors regard as the scope of the disclosure. Modifications of the above-described modes for carrying out the disclosure can be used by persons of skill in the art, and are intended to be within the scope of the following claims.

It is to be understood that the disclosure is not limited to particular methods or systems, which can, of course, vary. It is also to be understood that the terminology used herein is for the purpose of describing particular embodiments only, and is not intended to be limiting.

A number of embodiments of the disclosure have been described. Nevertheless, it will be understood that various modifications may be made without departing from the spirit and scope of the present disclosure. Accordingly, other embodiments are within the scope of the following claims.

What is claimed is:

1. A catheter system for implantation through the skin of a patient, the catheter system comprising:
a channel comprising a length operable to extend through the skin of the patient;
wherein at least a portion of the length of the catheter is surrounded by a first porous membrane that is operable to receive tissue in-growth from the patient and enable indwelling of the catheter; and
the first porous membrane comprises pore sizes of between approximately 1-50 microns.

2. The catheter system of claim 1, wherein the porous membrane is bonded to an exterior of the channel by at least one of a thermoplastic, silicone, cyanoacrylate and a polymer adhesive.

3. The catheter system of claim 1, wherein the first porous membrane is bonded to an exterior of the channel in a manner that is operable to reduce the entry of bacteria and prevent infections associated with the implantation of the catheter.

4. The catheter system of claim 1, wherein the channel comprises at least one of a silicone, a polymer and a thermoplastic.

5. The catheter system of claim 1, further comprising a second porous membrane provided between the channel and the first porous membrane.

6. The catheter system of claim 5, wherein the second porous membrane comprises pore sizes of between approximately 0.01 and 5 microns to allow for delivery of a therapeutic agent to the patient.

7. The catheter system of claim 1, wherein the catheter is in communication with an implantable cellular encapsulation device comprising a lumen for housing at least one of cells and a therapeutic agent.

8. The catheter system of claim 1, wherein the channel is in fluid communication with an oxygen delivery device.

9. The catheter system of claim 7, wherein at least one of the catheter and the implantable cellular encapsulation device comprises an oxygen diffuser.

10. The catheter system of claim 9, wherein the diffuser comprises a super-hydrophobic membrane.

11. The catheter system of claim 10, wherein the super-hydrophobic membrane comprises at lest one of a polytetrafluroethylene, an expanded polytetrafluroethylene, a polymer, a thermoplastic polyurethane, and silicone.

12. The catheter system of claim 7, further comprising an over-pouch operable to retain a fluid and surround the encapsulation device.

13. A catheter system for implantation through the skin of a patient, the catheter system comprising:
a channel comprising a length operable to extend through the skin of the patient;
wherein at least a portion of the length of the catheter is surrounded by a first porous membrane that is operable to receive tissue in-growth from the patient and enable indwelling of the catheter;
the first porous membrane comprising pore sizes of between approximately 1-50 microns; and
wherein the first porous membrane is thermally bonded to the catheter.

14. A catheter system for implantation through the skin of a patient, the catheter system comprising:
a channel comprising a length operable to extend through the skin of the patient;
wherein at least a portion of the length of the catheter is surrounded by a first porous membrane that is operable to receive tissue in-growth from the patient and enable indwelling of the catheter;
the first porous membrane comprises pore sizes of between approximately 1-50 microns; and
a first subcutaneous implantable encapsulation device comprising a lumen and a vascularization membrane, and wherein the lumen is in fluid communication with the channel.

15. The catheter system of claim 14, further comprising a second subcutaneous implantable encapsulation device comprising a lumen in communication with the channel.

16. The catheter system of claim 15, wherein the catheter comprises a bifurcation to deliver fluid to the first and second subcutaneous implantable encapsulation devices.
